(19) United States
(12) Patent Application Publication
(10) Pub. No.: US 2019/0329050 A1
(43) Pub. Date: Oct. 31, 2019

(54) CLINICAL NEUROSTIMULATION CONTROLLER

(71) Applicant: ALEVA NEUROTHERAPEUTICS, Lausanne (CH)

(72) Inventors: Jean-Charles Montavon, Lausanne (CH); Alexandre Michalis, Lausanne (CH); Pascal Harbi, Lausanne (CH)

(21) Appl. No.: 15/962,632

(22) Filed: Apr. 25, 2018

(51) Int. Cl. A61N 1/372; A61N 1/36; A61N 1/05

(52) U.S. Cl. CPC A61N 1/37247; A61N 1/36135; A61N 1/0534; G16H 40/67; A61N 1/3615; A61N 1/36175; A61N 1/36132

(57) ABSTRACT

The present disclosure describes system and methods to configure an implantable neurostimulation device. The system can include a programmer for neurostimulation devices. The programmer can be a handheld device that programs the stimulation parameters for both new and existing patients. The programmer can configure the neurostimulation device to iteratively deliver stimulations through each of the lead's electrodes. The programmer can receive and record indications of the patient's response to each of the stimulations and generate benefit scores or side effect scores based on the patient's response. The programmer can determine the scores based on data received from patient monitors, external sensors, and clinician input. Based on the scores, the programmer can generate therapeutic windows for each of the electrodes. The programmer can combine the therapeutic windows into a therapeutic window map.

### BACKGROUND

Deep brain stimulation (DBS) can include neurostimulation therapy that involves electrical stimulation systems that stimulate the human brain and body. DBS can be used to treat a number of neurological disorders. DBS can involve electrically stimulating a target area of the brain. Different stimulation parameters can be selected for each of the electrodes used in DBS and other stimulation paradigms. The stimulation parameters can include a number of independently controlled variables, such as frequency, duration, and intensity.

### SUMMARY

According to at least one aspect of the disclosure, a system to select stimulation electrodes can include a data processing system. The data processing system can include one or more processors and a memory that execute an interface, a communication component, a scoring component, and a mapping component. The system can receive and record an indication of a configuration of a neurological lead. The neurological lead can include a plurality of electrodes. The system can receive and record an implantation location of the neurological lead. The system can transmit to an implanted stimulation device a first message to deliver a first stimulation signal to one of the plurality of electrodes. The first stimulation signal can have a first set of stimulation parameters. The system can transmit to the implanted stimulation device a second message to deliver a second stimulation signal to the one of the plurality of electrodes. The second stimulation signal can have a second set of stimulation parameters. The system can receive and record an indication of a first stimulation effect based on the first stimulation signal to the one of the plurality of electrodes. The system can receive and record an indication of a second stimulation effect based on the second stimulation signal to the one of the plurality of electrodes. The system can determine a therapeutic window for the one of the plurality of electrodes based differences between a first and second set of stimulation parameters, the indication of the first stimulation effect, and the indication of the second stimulation effect. The system can generate a therapeutic window map based on the therapeutic window for the one of the plurality of electrodes, the indication of the configuration of the neurological lead, and the implantation location of the neurological lead.

According to at least one aspect of the disclosure, a method to select stimulation electrodes of an implantable neurostimulation device can include receiving an indication of a configuration of a neurological lead. The neurological lead can include a plurality of electrodes. The method can include receiving an implantation location of the neurological lead. The method can include transmitting, to an implanted stimulation device, a first message to deliver a first stimulation signal to one of the plurality of electrodes. The first stimulation signal can have a first set of stimulation parameters. The method can include transmitting, to the implanted stimulation device, a second message to deliver a second stimulation signal to the one of the plurality of electrodes. The second stimulation signal can have a second set of stimulation parameters. The method can include receiving an indication of a first stimulation effect based on the first stimulation signal to the one of the plurality of electrodes. The method can include receiving an indication of a second stimulation effect based on the second stimulation signal to the one of the plurality of electrodes. The method can include determining a therapeutic window for the one of the plurality of electrodes based on a difference between the first and second set stimulation parameters, the indication of the first stimulation effect, and the indication of the second stimulation effect. The method can include generating a therapeutic window map based on the therapeutic window for the one of the plurality of electrodes, the indication of the configuration of the neurological lead, and the implantation location of the neurological lead.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 illustrates a system to program and configure an implantable neurostimulation device.

FIG. 2 illustrates a graphical user interface that can be generated by the system illustrated in FIG. 1.

FIG. 3 illustrates a graphical user interface that can be generated by the system illustrated in FIG. 1.

FIG. 4 illustrates a block diagram of an example method to select stimulation electrodes of an implantable neurostimulation device using the system illustrated in FIG. 1.

FIG. 5 illustrates an example therapeutic window map generated by the system illustrated in FIG. 1.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

Programming an implantable neurostimulation device can include selecting which electrodes of a lead should be used for stimulation and selecting the stimulation parameters for each of the selected electrodes. The stimulation parameters can include a number of different options such as stimulation current, voltage, frequency, duration, and duty cycle. Each of the different parameters, delivered at each of the different electrodes, can cause different levels of benefit or side effect to the patient. Accordingly, the programming of a multi-electrode lead can be time-consuming for both the clinician and patient because so many options need to be evaluated. The time requirement continues to increase as leads continue to increase their electrode count.

The present disclosure describes a programmer for neurostimulation devices. The programmer can be a handheld device that generates a graphical user interface that provides a clear work-flow to program the stimulation parameters for both new and existing patients. The programmer can configure the neurostimulation device to iteratively deliver stimulations through each of the lead's electrodes. The programmer can automatically select the stimulation protocol or can set the stimulation protocol based at least one input from a clinician. The programmer can receive and record indications of the patient's response to each of the stimulations and generate benefit scores or side effect scores based on the patient's response. The programmer can determine the scores based on data received from patient monitors, external sensors, and clinician input. Based on the scores, the programmer can generate therapeutic windows for each of the electrodes. The programmer can combine the therapeutic windows into a therapeutic window map. Based on the therapeutic window map, electrodes and corresponding stimulation parameters that can provide the most benefit to the patient are selected to deliver therapeutic stimulation treatments. The programmer can automate and provide work-flows for the selection of stimulation parameters, which can enable evidence-based selection of the electrodes and stimulation parameters.

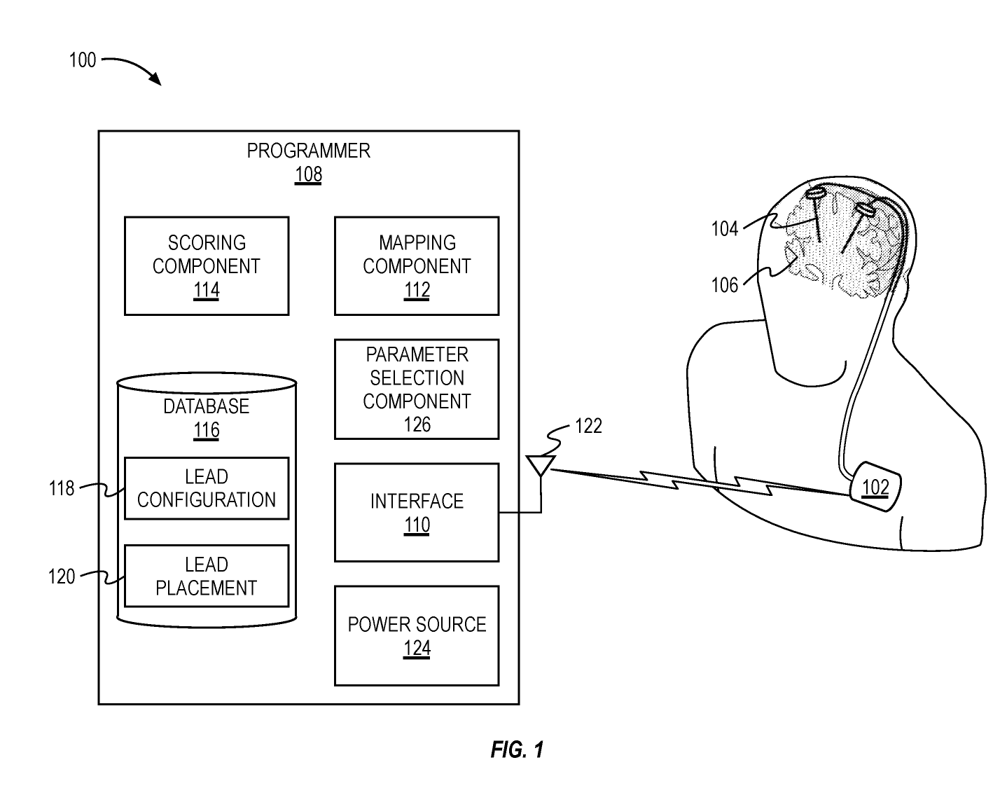

FIG. 1 illustrates a system 100 to program and configure an implantable neurostimulation device 102. The neurostimulation device 102 can provide electrical stimulation to and receive electrical signals from the patient's brain 106 via the leads 104. The programmer 108 can program and configure the neurostimulation device 102. The programmer 108 can include an interface 110, a mapping component 112, and a scoring component 114. The programmer 108 can communicate with the neurostimulation device 102 via the interface 110. The interface 110 can include or can interface with an antenna 122. The programmer 108 can include a power source 124. The programmer 108 can include a parameter selection component 126. The programmer 108 can include a database 116. Data files that include lead configurations 118 and lead placements 120 can be stored in the database 116.

The neurostimulation device 102 can be an implantable stimulation device. The neurostimulation device 102 can be a hermetically sealed device that includes a plurality of electrical components for the generation of electrical pulses and the recording of electrical signals. The neurostimulation device 102 can include a power source, such as a battery, that enables the neurostimulation device 102 to generate electrical stimulation pulses that are delivered to the leads 104 via cables and then to the patient via electrodes. The electrical stimulation pulses can travel through the leads 104 and into the brain 106 (or other tissue). The stimulation pulse's current, voltage, frequency, duration, duty cycle, and through which electrodes of the lead 104 the stimulation pulse is delivered can be configured by the programmer 108. The neurostimulation device 102 can include a memory element to which the configurations from the programmer 108 are stored. The neurostimulation device 102 can include a plurality of analog to digital converters that enable electrical signals generated by the brain 106 (or implanted tissue) to be detected and digitized. The neurostimulation device 102 can store the digitized signals to the memory element. The neurostimulation device 102 can intermittently or continuously establish a data connection with the programmer 108 to transmit and receive data with the programmer 108. For example, the programmer 108 can provide the neurostimulation device 102 with updated stimulation parameters and the neurostimulation device 102 can provide the programmer 108 with recently recorded electrical signals from the brain 106.

The lead 104 can be any neurological lead or other lead that can be used to detect or deliver electrical signals to or from the tissue. The lead 104 can include a plurality of electrodes. The lead 104 can be configured for chronic or acute implantation. The lead 104 can be a lead such as that described in U.S. Pat. No. 9,474,894, which is incorporated by reference in its entirety. For example, the lead 104 can be a multidirectional, deep-brain stimulation lead. The lead's distal end can include a flexible microelectromechanical system (MEMS) film. The MEMS film can include a plurality of electrodes. The electrodes can be positioned circumferential around the lead's distal end. The lead's distal end can include one or more electrodes at different axial positions.

The programmer 108 can be a data processing system that can include one or more processors. The programmer 108 can be desktop computer, a laptop computer, a handheld computer, a tablet device, mobile phone, client device, or other computing platform. The programmer's processors can execute the interface 110, mapping component 112, parameter selection component 126, and scoring component 114. The programmer's processors can include one or more of a digital processor, an analog processor, a digital circuit designed to process information, an analog circuit designed to process information, a state machine, and/or other mechanisms for electronically processing information. One or more functions of the programmer 108 can be performed by a data processing system. For example, the programmer 108 can be a tablet device and the functions of the mapping component 112 can be performed by a desktop computer. The programmer 108 can also interface with external resources that can include, for example, networked or other forms of remote storage. The programmer 108 can access the remote storage to download patient details to the database 116 when programming the patient's neurostimulation device 102. The programmer 108 can also interface with other medical devices such as patient monitors, heart rate monitors, cameras, and external sensors (e.g., accelerometers).

The programmer 108 can include an interface 110. The interface 110 can be a physical (e.g., hardware) interface or a software interface. The interface 110 can be a software interface. The interface 110 can generate a graphical user interface that enables a user to interact with the programmer 108. The interface 110 can also provide one or more application programming interfaces (APIs) that enable other components, devices, or software to interact with the programmer 108. The interface 110 can be a physical interface. The physical interface can be a data, network, or wireless connection that enables a user and other devices to interact with the programmer 108. The programmer 108 can receive and transmit data from the neurostimulation device 102 via the interface 110. For example, the interface 110 can include an antenna 122. The antenna 122 can enable the programmer 108 to wirelessly interface with the neurostimulation device 102.

The programmer 108, via the interface 110, can be configured to receive and record an indication of a configuration of the lead 104. The lead configuration 118 can be a file or data that can include lead type, manufacturer, electrode count, lead shape, electrode positions, and other information about the lead 104. The electrode positions can indicate the angular position of the lead's electrodes and the depth (or distance from the distal tip) of each of the electrodes. The interface 110 can interface with patient records to retrieve the lead configuration 118 from the patient's records. The interface 110 can provide a graphical user interface that enables a user to select or enter the lead configuration 118. The interface 110 can save the lead configuration 118 to the database 116.

The programmer 108, via the interface 110, can be configured to receive a data file that includes lead placement 120. The lead placement 120 can indicate the implantation location, implantation depth, implantation coordinates, and lead orientation (which can be an angular position or an angle of insertion into the tissue). The interface 110 can interface with patient records to retrieve the lead placement 120 from the patient's records. The interface 110 can provide a graphical user interface that enables a user to select or enter the lead placement 120. The interface 110 can save the lead placement 120 to the database 116.

The programmer 108 can include the parameter selection component 126. The parameter selection component 126 can select stimulation and recording parameters for the neurostimulation device 102 and lead 104. The parameter selection component 126 can select the stimulation and recording parameters and generate messages containing the parameters. The messages can be transmitted to the neurostimulation device 102 via the interface 110. The neurostimulation device 102 can receive and process the message to set the parameters at the neurostimulation device 102. The parameters can include stimulation voltage amplitude, stimulation current amplitude, stimulation frequency, stimulation duty cycle, stimulation duration, and electrode configuration. The electrode configuration can indicate whether the electrode is active, inactive, configured as a recording electrode, configured as a stimulating electrode, or configured to switch between a stimulating and recording electrode. For example, the message the parameter selection component 126 generates can indicate to the neurostimulation device 102 how each of the lead's electrodes should be configured and the intensity (as measured by voltage and/or current) of the stimulation that should be delivered to each electrode that is configured as a stimulating electrode.

During a mapping phase where therapeutic windows for each electrode are determined, the parameter selection component 126 can generate a plurality of messages that are transmitted to the neurostimulation device 102. The different messages can include different configurations that are sequentially transmitted and applied to the neurostimulation device 102. For example, the parameter selection component 126 can generate a plurality of messages that cause the neurostimulation device 102 to sequentially generate a greater intensity stimulation signal that is delivered to the brain 106 via the lead 104. During the mapping phase, the parameter selection component 126 can also generate a single message that includes a plurality of configurations. For example, the parameter selection component 126 can generate a message that causes the neurostimulation device 102 to periodically increase a stimulation parameter, such as the stimulation intensity, of subsequent stimulation pulses.

The parameter selection component 126 can also set the therapeutic stimulation parameters. The therapeutic stimulation parameters can be parameters that are selected by the parameter selection component 126 after the mapping phase. The therapeutic stimulation parameters can be selected based on input from a clinician. The neurostimulation device 102 can be configured with the therapeutic stimulation parameters until the neurostimulation device 102 is reprogrammed or another mapping phase is completed. When selecting the therapeutic stimulation parameters, the parameter selection component 126 can select which of the lead's electrodes will be configured as stimulating electrodes and the stimulation intensity to be delivered by each of the stimulating electrodes. The parameter selection component 126 can select to configure the neurostimulation device 102 to deliver the same or different stimulation intensities to each of the stimulation electrodes. The parameter selection component 126 can select the therapeutic stimulation parameters based on the therapeutic window map that is generated by the mapping component 112 during the mapping phase.

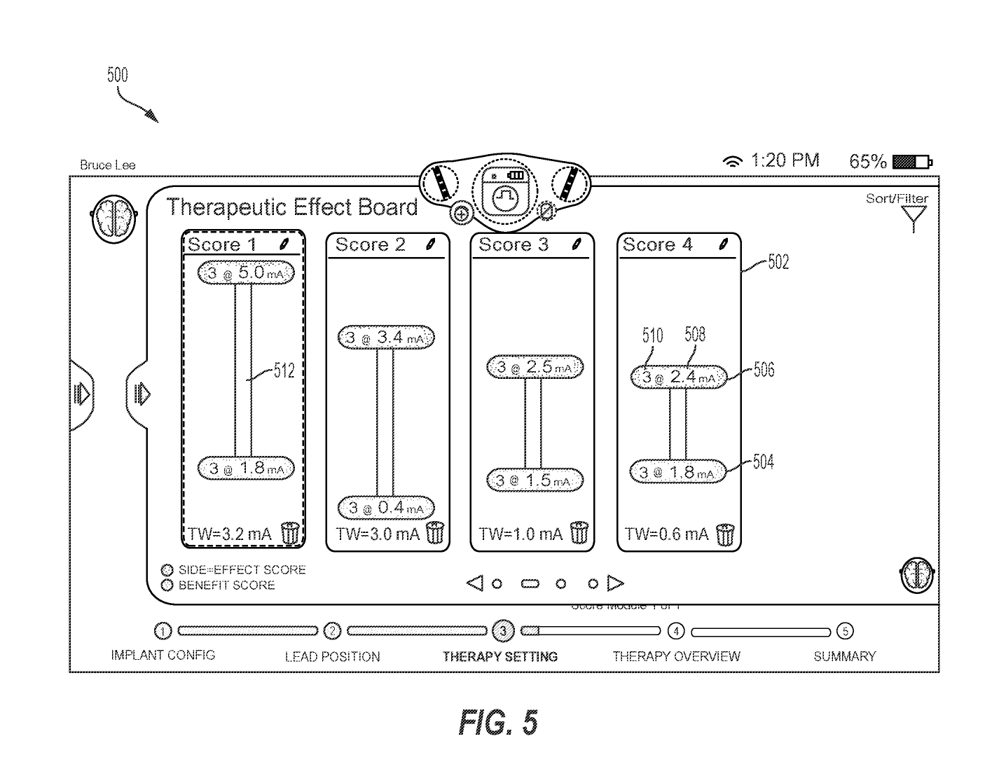

The programmer 108 can include a mapping component 112 that can generate therapeutic window maps. The therapeutic window maps can be displayed to a user via the interface 110. The therapeutic window maps can be used to select which of the lead's electrodes should be used for stimulation and the parameters of the stimulation signal that should be applied through the selected stimulation electrodes. The therapeutic window maps are described further in relation to FIG. 5, among others.

The mapping component 112 can determine therapeutic windows for each of the lead's electrodes. An electrode's therapeutic window can indicate the range of intensities over which the electrode has a stimulation effect on the patient. The stimulation effect can be beneficial (e.g., reduces symptoms) or negative (e.g., causes side effects). For example, the therapeutic window for an electrode can be defined between a stimulation intensity where a therapeutic benefit is first detected and the stimulation intensity where a side effect is first detected or becomes intolerable. A negative stimulation effect can also occur when a given stimulation parameter provides reduced symptom reduction when compared to stimulations with a lower intensity. Each electrode can have a different therapeutic window based on the electrode's placement within the target tissue. For example, a first electrode placed relatively far from the target site may have no therapeutic window because stimulation from the electrode may never generate a stimulation effect. A second electrode placed relatively near the target site may have a large therapeutic window because a stimulation effect can be generated by providing a low intensity stimulation at the electrode and a side effect may not occur until a relatively high intensity stimulation is applied to the electrode.

During the mapping phase, the mapping component 112 can instruct the parameter selection component 126 to select a plurality of stimulation parameters. The parameter selection component 126 can configure or instruct the neurostimulation device 102 to iteratively apply stimulation signals at the plurality of stimulation parameters to a selected electrode. For example, a first stimulation may have a first voltage or current level and a second, subsequent stimulation may have a second voltage or current level. The second voltage or current level can be higher than the first voltage or current level. As described further in relation to the scoring component 114, the scoring component 114 can determine a stimulation effect for each of the stimulation parameters. The mapping component 112 can generate a therapeutic window for the selected electrode based at least on the stimulation effects determined by the scoring component 114.

The mapping component 112 can determine a minimum response stimulation parameter for each of the plurality of electrodes and a maximum response stimulation parameter for each of the plurality of electrodes. The therapeutic window for each electrode can be based on the minimum response stimulation parameter and the maximum response stimulation parameter. The minimum response stimulation parameter can be the stimulation parameters (e.g., stimulation current) at which a therapeutic benefit is first detected. The maximum response stimulation parameter can be the stimulation parameters at which a side effect is first detected or when an increase in stimulation intensity is no longer correlated with an increase in therapeutic benefit. The mapping component 112 can generate a therapeutic window map based on the therapeutic window for one or more of the electrodes. The therapeutic window map can be generated based on the therapeutic window for each of the plurality of electrodes or a sub-portion of the electrodes.

The programmer 108 can include a scoring component 114. The scoring component 114 can determine the stimulation effect of the stimulation signals applied via the selected electrodes. The scoring component 114 can determine if the stimulation generated a benefit or a side effect. The scoring component grades or scores the negative or positive stimulation effect of each stimulation signal. The score can be based on the Unified Parkinson's Disease Rating Scale.

The scoring component 114 can determine the stimulation effect based on each of the stimulation signals applied to each of the electrodes. The scoring component 114 can determine the stimulation effect of a stimulation signal based on input provided by a user, data received from a secondary device, or data received from a sensor. For example, to measure the effect a stimulation signal has on a Parkinson's patient, the scoring component 114, via the interface 110, can interface with one or more accelerometers located on the patient's hands. The accelerometers can measure the tremors of the patient's hands. The accelerometers can measure the decrease (or increase) in the tremors as different stimulation signals are delivered to the patient. In this example, the scoring component 114 can determine whether the stimulation generated a benefit when a decrease in tremor movement is detected. The scoring component 114 can determine when a stimulation cased a side effect. For example, the scoring component 114 can interface with a heart rate monitor. The scoring component 114 can detect changes in the patient's heart rate, which the scoring component 114 can classify as a side effect. The scoring component 114 can also detect tremors by analyzing video data of the patient as the stimulation is applied to the patient. The scoring component 114 can detect benefits and side effects based on data provided by a user. For example, the patient can self-report to the programmer 108 and provide assessments to the scoring component 114 after the application of a stimulation. The scoring component 114 can also use input from a medical professional to determine the presence of a benefit or side effect.

The scoring component 114 can combine the stimulation effect data to generate a benefit score or a side effect score. The benefit score can indicate the relative degree to which the stimulation provided a therapeutic benefit. The side effect score can indicate the relative degree to which the stimulation caused a side effect in the patient. The scoring component 114 can generate both a benefit score and a side effect score for a stimulation signal. For example, a stimulation can reduce symptoms but also cause a side effect.

The programmer 108 can include a database 116. The database 116 can be any form of electronic storage. For example, the electronic storage can include non-transitory storage media that electronically stores information or data. The electronic storage media can include one or both of storage internal to the programmer 108 or storage located remote to the programmer 108. The remote storage can couple with the programmer 108 via the interface 110 (e.g., through a USB port, a firewire port, network port, etc.). The programmer 108 can communicate with the remote storage through a physical connect (e.g., a physical network connection or USB cable) or wirelessly (e.g., through a wireless network connection). The electronic storage may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storage may include one or more virtual storage resources (e.g., cloud storage, a virtual private network, and/or other virtual storage resources).

The programmer 108 can store data files into the database 116. The data files can include lead configurations 118 and lead placements 120. The programmer 108 can generate separate lead configurations 118 and lead placements 120 for each patient.

The lead configurations 118 can be data structures or data files that can indicate the type and manufacturer of lead 104 and neurostimulation device 102. The lead configurations 118 can indicate the number of electrodes each lead 104 contains and how the electrodes are placed or distributed on the lead 104. The lead configuration 118 can indicate what type, shape, and size of the lead's electrodes.

The lead placements 120 can be data structures or data files that can indicate the placement or location of the lead 104 (and it's electrodes) within the patient's tissue, such as the brain 106. The lead placements 120 can indicate the lead's location, depth, rotation, and angle of insertion. The placement location information can be determined by a surgeon using stereotactic tools during the implantation of the leads 104. The placement location information can be determined through post-operative imagining. The programmer 108 can retrieve the lead placements 120 from the patient's medical data files or a user of the programmer 108 can enter the lead placement 120 data.

The programmer 108 can include a power source 124. The power source 124 can be a battery. The battery can be rechargeable. The power source 124 can be a power converter that enables the programmer 108 to couple with wall power and the power source 124 can convert the wall power's alternating current into direct current.

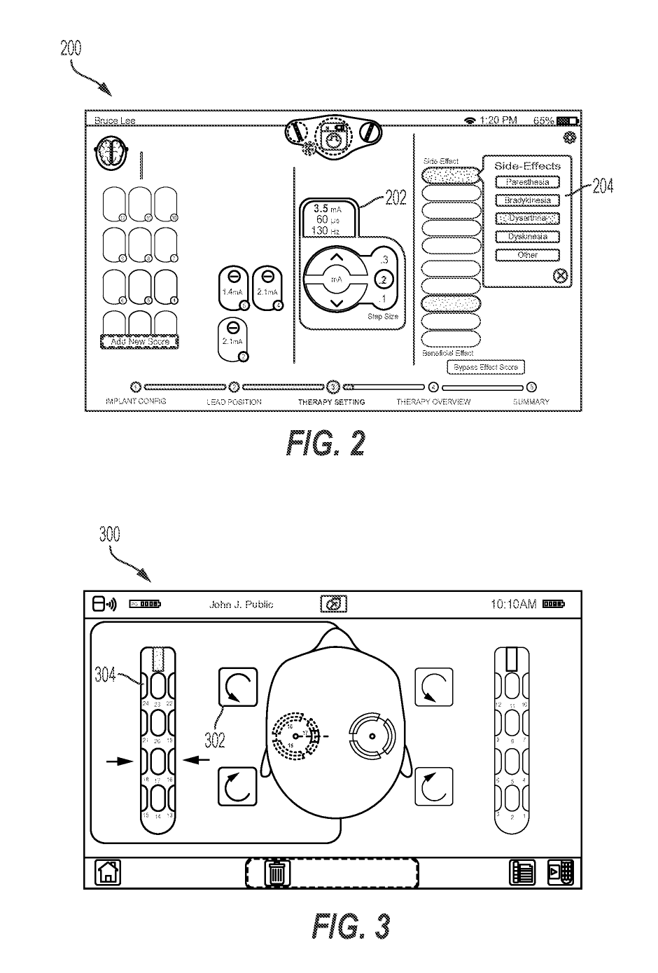

FIG. 2 illustrates a graphical user interface (GUI) 200 that can be generated by the programmer 108. Referring also to FIG. 1, the GUI 200 can be generated by the interface 110. The interface 110 can include data or functions from the mapping component 112, scoring component 114, and parameter selection component 126 to generate the GUI 200.

The GUI 200 can include a stimulation widget 202 that includes data from and can be controlled by the parameter selection component 126. Via the stimulation widget 202 the user can set the step size between stimulation pulses. The step size can indicate the increase (or decrease in the case of a negative step) in current or voltage that should occur between subsequent stimulation pulses. For example, if the step size is 0.2 mA, a first stimulation pulse may be 3.0 mA and a second stimulation pulse may be 3.2 mA. A user can also set the minimum and maximum stimulation parameters via the stimulation widget 202. The user also set the duty cycle and frequency of the stimulation pulses. If the stimulation includes a pulse train, the duty cycle can indicate the time between the pulses of the pulse train. The frequency can indicate the stimulation frequency of a pulse. For example, each pulse of a pulse train may be delivered at 130 Hz (the frequency) with an inter-pulse spacing of 60 microseconds (the duty cycle). In some implementations, the parameter selection component 126 can automatically supply the stimulation parameters to the stimulation widget based on feedback from the scoring component 114 or a user can manually enter the stimulation parameters.

The GUI 200 can include an effect widget 204. The effect widget 204 can include data from and be controlled by the scoring component 114. The effect widget can illustrate to a user at what stimulation parameters the patient first experienced therapeutic benefit and at what stimulation parameters the patient first experienced a side effect. The effect widget 204 can indicate the side effects to the user.

FIG. 3 illustrates a GUI 300 that can be generated by the programmer 108. Referring also to FIG. 1, the GUI 300 can be generated by the interface 110. The interface 110 can include data or functions from the mapping component 112 and the database 116. The GUI 300 can illustrate to a user the general placement of the lead 104 within the patient. The GUI 300 can illustrate the general configuration of the lead 104. The placement and configuration of the lead 104 can be retrieved from the lead configuration 118 and lead placement 120 files within the database 116.

The GUI 300 can also enable a user to input the lead configuration 118 and lead placement 120 information into the programmer 108. For example, the GUI 300 can include buttons 302 that enable the user to rotate the lead representation 304 so that the lead representation 304 corresponds to the proper orientation of the lead 104 within the patient. Via the GUI 300, the user can also enter lead position, location, and depth information.

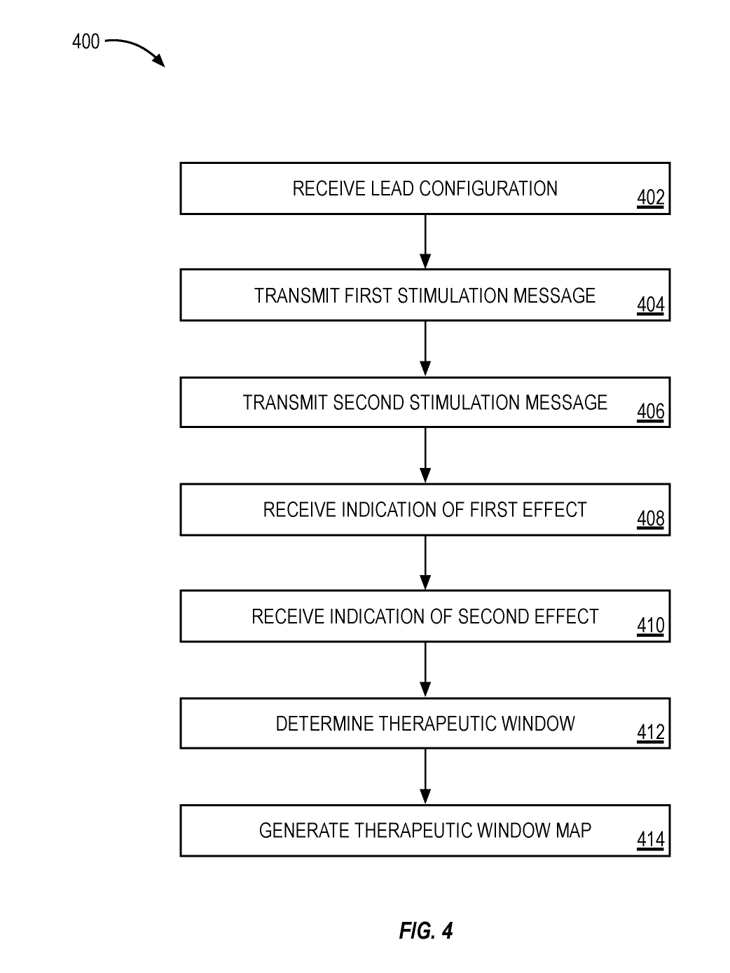

FIG. 4 illustrates a block diagram of an example method 400 to select stimulation electrodes of an implantable neurostimulation device. The method 400 can include receiving lead configurations (ACT 402). The method 400 can include transmitting a first stimulation message (ACT 404) and a second stimulation message (ACT 406). The method 400 can include receiving an indication of a first effect (ACT 408) and the indication of a second effect (ACT 410). The method 400 can include determining a therapeutic window (ACT 412). The method 400 can include generating a therapeutic window map (ACT 414). Also referring to FIG. 1, among others, the method 400 can be performed by the programmer 108.

As set forth above, the method 400 can include receiving an indication of a configuration of a neurological lead (ACT 402). The programmer 108 can automatically retrieve the configuration of the lead 104 for a patient file. For example, a user can input the patient's name or identifier into the programmer 108 and the programmer 108 can retrieve the lead configuration 118 from the patient's file or database 116. The programmer 108 can present a GUI to the user that enables a user to manually enter or review the lead configuration 118. The indication of the configuration of the neurological lead can be the lead configuration 118 and can include at least the lead 104 type, configuration, electrode count, and electrode configuration.

The ACT 402 of retrieving the lead configuration can also include retrieving or receiving the lead placement 120. The programmer 108 can automatically retrieve the lead placement 120 of the 104 for the patient's file. For example, a user can input the patient's name or identifier into the programmer 108 and the programmer 108 can retrieve the lead placement 120 from the patient's file or database 116. The programmer 108 can present a GUI to the user that enables a user to manually enter or review the lead placement 120. The lead placement 120 can include lead position, orientation, depth, and other position information.

The method 400 can include transmitting a first stimulation message (ACT 404). For example, the method 400 can include transmitting, to an implanted stimulation device (such as the neurostimulation device 102), a first message to deliver a first stimulation signal to at least one of the lead's electrodes. For example, the parameter selection component 126 can select stimulation parameters that can include stimulation intensity (as measured by voltage and/or current), frequency, and duty cycle. The parameter selection component 126 can generate a message that includes the stimulation parameters. The message can be transmitted to the neurostimulation device 102 via the interface 110 and antenna 122. Responsive to receiving the message, the neurostimulation device 102 can deliver a stimulation to the one or more electrodes indicated in the message. The message can instruct or configure the neurostimulation device 102 deliver a stimulation pulse to one, more than one, or all of the lead's electrodes. When the message instructs or configures the neurostimulation device 102 to deliver a stimulation pulse to multiple electrodes, the neurostimulation device 102 can deliver stimulations to each of the selected electrodes that are different or the same.

The method 400 can include transmitting a second stimulation message (ACT 406). For example, the method 400 can include transmitting, to the implanted stimulation device, a second message to deliver a second stimulation signal to the one or more of the lead's plurality of electrodes. The parameter selection component 126 can select the stimulation parameters that are included in the second stimulation message. The parameter selection component 126 can increment the stimulation intensity by a positive or negative step size and include the updated stimulation intensity in the second message. The second message can be transmitted to the neurostimulation device 102 via the interface 110 and antenna 122. The second stimulation message can be a component of the first message or sent with the first message. For example, the parameter selection component 126 can generate a stimulation message that indicates a test protocol and the stimulation to be delivered by the neurostimulation device 102 during the test protocol.

The method 400 can include receiving an indication of a first effect (ACT 408) and an indication of a second effect (ACT 410). The first stimulation effect can be based on the first stimulation signal delivered to the patient responsive to the message transmitted at ACT 404. The second stimulation effect can be based on the second stimulation signal delivered to the patient responsive to the message transmitted at ACT 406. The stimulation effect can be a therapeutic benefit, a side effect, or no effect. The stimulation effect can also be scored to indicate a degree of the effect. An indication of the stimulation effect can be entered into the programmer 108 via the interface 110 by a user. For example, the interface 110 can provide a GUI to the user that provides the user with a plurality of options to rank, grade, or classify the stimulation effect. The programmer 108 can also automatically determine the stimulation effect via the scoring component 114. For example, the scoring component 114 can receive data from patient monitors that can include heart rate monitors, blood pressure monitors, respiration monitors, and temperature monitors; imaging devices that can include still or video imaging devices; and motion sensors that can include accelerometers. Based on, for example, a decrease in the patient's tremor (as measured by an accelerometer), the scoring component 114 can determine that a therapeutic benefit occurred responsive to the stimulation. In another example, the scoring component 114 can determine that a side effect occurred based on at least detecting a decrease in the patient's heart rate following or during the stimulation.

The scoring component 114 can compare detected effects to a threshold before classifying the effect as a stimulation effect. For example, a decrease in heart rate may not be classified as a stimulation effect constituting a side effect until the heart rate decreases 10% from the patient's baseline resting heart rate. The scoring component 114 can use data from external sensors, sources, input from a user, or any combination thereof to determine and classify the stimulation effect. The stimulation and detection of stimulation effects can be referred to as the mapping phase for an electrode.

The delivery of stimulation messages with updated stimulation parameters and the detection of stimulation effects can be repeated a plurality of times for each of the lead's electrodes. For example, for each electrode, the parameter selection component 126 can configure the neurostimulation device 102 to iteratively increase and deliver stimulation pulses until a maximum stimulation intensity or side effect is reached. The programmer 108 can determine, receive, or record a stimulation effect for each of the stimulation pulses. In some implementations, the mapping phase may be conducted on only a portion of the lead's electrodes. For example, based on the lead configuration 118 and the lead placement 120 the programmer 108 may not perform the mapping phase on electrodes that are not near the target area or on electrodes that are directed toward brain regions known to cause side effects when stimulated. The programmer 108 can determine the order at which each electrode is mapped. The programmer 108 can order the electrode mapping phases based on which electrode is expected to provide the relatively highest therapeutic benefit. For example, the programmer 108 can first select electrodes placed relatively near the target region and later select (or not select at all) electrodes that are placed away from the target region or near regions known to cause side effects.

The method 400 can include determining a therapeutic window (ACT 412). For example, the programmer 108 can determine a therapeutic window for an electrode based on the indication of the first stimulation effect and the indication of the second stimulation effect. The therapeutic window can be based on the differences in the stimulation parameters (e.g., the stimulation intensities) between the stimulation that cause the first stimulation effect and the second stimulation effect. The therapeutic window can be a data structure generated by the mapping component 112 that is stored in the database 116. The therapeutic window can include the stimulation parameters (e.g., intensity, frequency, and duty cycle) at which a therapeutic benefit was first detected. The therapeutic window can include the stimulation parameters at which a side effect was first detected. The therapeutic window can store a score, as generated by the scoring component 114, that can indicate a degree or intensity of the therapeutic benefit or side effect. The mapping component 112 can generate a therapeutic window for each of the lead's electrodes.

The method 400 can include generating a therapeutic window map (ACT 414). For example, the programmer 108 can generate a therapeutic window map based on the therapeutic window (or windows) calculated during the above-described ACTs of method 400. The therapeutic window map can include a visual representation of one or more of the electrodes therapeutic windows. The therapeutic window map can visually represent the therapeutic windows for each of a plurality of selected electrodes. Therapeutic window maps are described further in relation to FIG. 5. The therapeutic window maps can provide a visual representation of the differences between the stimulation parameters where a benefit effect was detected and the stimulation parameters where a side effect was detected.

The method 400 can also include selecting therapeutic stimulation parameters. The therapeutic stimulation parameters can be the stimulation parameters that the programmer 108 configures the neurostimulation device 102 to deliver to the patient during treatment. The programmer 108 or clinician can select which electrodes to use based at least on, for example, the size of each electrodes therapeutic envelop 512. For example, the programmer 108 can select the electrodes with the greatest separation between the benefit level 504 and the side effect level 506. The selection of the electrodes and the stimulation parameters can also be based on the scores 510 for each of the benefit levels 504 and side effect levels 506.

FIG. 5 illustrates an example therapeutic window map 500. The therapeutic window map 500 can include a therapeutic range 502 for each of the lead's electrodes (or a portion thereof). As illustrated in FIG. 5, the therapeutic window map 500 includes a therapeutic range 502 for four electrodes. Each therapeutic range 502 can include a benefit level 504 and a side effect level 506. Each of the benefit level 504 and the side effect level 506 can include a score 510 and a stimulation intensity 508. The benefit level 504 can indicate the stimulation intensity 508 at which the scoring component 114 first detected or determined there to be a therapeutic benefit. The side effect level 506 can indicate the stimulation intensity 508 at which the scoring component 114 first detected or determined there to be a side effect. The side effect level 506 can indicate the stimulation intensity 508 at which the scoring component 114 detected the largest or a significant side effect. For example, a relatively lower stimulation intensity may cause a side effect that is acceptable to the patient and a relatively higher stimulation intensity may cause a side effect that the patient cannot tolerate. The scoring component 114 can set the side effect level 506 the relatively higher stimulation intensity rather than the relatively lower intensity. The benefit level 504 and the side effect level 506 can also include a score 510. The score 510 can be determined by the scoring component 114. The score 510 can indicate the degree, intensity, or grade of the therapeutic benefit and side effect.

The therapeutic range 502 can also visually indicate the therapeutic envelop 512. The therapeutic envelop 512 can visually indicate the distance between the benefit level 504 and the side effect level 506. For example, as illustrated in FIG. 5, the first electrode has a benefit level 504 at 1.8 mA and a side effect level 506 at 5 mA. The therapeutic envelop 512 of the first electrode is 3.2 mA. The therapeutic window map 500 can provide a visual representation that enables medical professional to select which electrodes and at what stimulation parameters to deliver therapeutic stimulation. The therapeutic window map 500 can enable medical professionals to compare electrodes and enable the medical profession to select electrodes with large therapeutic envelops 512. The mapping component 112 can normalize the length of the bar representing therapeutic envelop 512 based on which therapeutic ranges 502 are displayed on the therapeutic window map 500. For example, the mapping component 112 can calculate a stimulation range for each of the electrodes represented in the therapeutic window map 500 by determining the difference between the side effect level's stimulation intensity 508 and the benefit level's stimulation intensity 508. To normalize the stimulation ranges, each stimulation range can be divided by the stimulation range with the largest magnitude. The length of the therapeutic envelop 512 can be calculated based on the normalized stimulation range.

While operations are depicted in the drawings in a particular order, such operations are not required to be performed in the particular order shown or in sequential order, and all illustrated operations are not required to be performed. Actions described herein can be performed in a different

The separation of various system components does not require separation in all implementations, and the described program components can be included in a single hardware or software product.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

As used herein, the term “about” and “substantially” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation or embodiment, and references to “an implementation,” “some implementations,” “one implementation” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

The indefinite articles “a” and “an,” as used herein in the specification and in the claims, unless clearly indicated to the contrary, should be understood to mean “at least one.”

References to “or” may be construed as inclusive so that any terms described using “or” may indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

1. A system to select stimulation electrodes with a data processing system comprising one or more processors and a memory that execute an interface, a communication component, and a mapping component to:
receive, by the interface, an indication of a configuration of a neurological lead, the neurological lead comprising a plurality of electrodes;
transmit, by the communication component and to an implanted stimulation device, a first message to deliver a first stimulation signal with a first set of stimulation parameters to one of the plurality of electrodes;
transmit, by the communication component and to the implanted stimulation device, a second message to deliver a second stimulation signal with a second set of stimulation parameters to the one of the plurality of electrodes;
receive, by an interface, an indication of a first stimulation effect based on the first stimulation signal to the one of the plurality of electrodes;
receive, by an interface, an indication of a second stimulation effect based on the second stimulation signal to the one of the plurality of electrodes;
determine, by the mapping component, a therapeutic window for the one of the plurality of electrodes based on a difference between the first set of stimulation parameters and the second set of stimulation parameters, the indication of the first stimulation effect, and the indication of the second stimulation effect; and
generate, by the mapping component, a therapeutic window map based on the therapeutic window for the one of the plurality of electrodes and the indication of the configuration of the neurological lead.

2. The system of claim 1, wherein the first stimulation signal has a first current level and the second stimulation signal has a second current level different than the first current level.

3. The system of claim 1, wherein the first stimulation signal has a first voltage level and the second stimulation signal has a second voltage level different than the first voltage level.

4. The system of claim 1, comprising a scoring component to:
determine a benefit score for each of at least the portion of the plurality of electrodes based on the minimum response stimulation parameter; and
determine a side effect score for each of at least the portion of the plurality of electrodes based on the maximum response stimulation parameter.

5. The system of claim 1, comprising:
the communication component to transmit, to the implanted stimulation device, a third message to increase a stimulation parameter to the one of the plurality of electrodes.

6. The system of claim 1, comprising the mapping component to:
determine a minimum response stimulation parameter for each of at least a portion of the plurality of electrodes;
determine a maximum response stimulation parameter for each of at least the portion of the plurality of electrodes; and
determine a therapeutic window for each of at least the portion of the plurality of electrodes based on the minimum response stimulation parameter for each of at least the portion of the plurality of electrodes and the maximum response stimulation parameter for each of at least the portion of the plurality of electrodes.

7. The system of claim 6, comprising:
the mapping component to generate the therapeutic window map based on the therapeutic window for each of at least the portion of the plurality of electrodes.

8. The system of claim 6, comprising:
the mapping component to set at least one of the plurality of electrodes as a stimulation electrode based on the therapeutic window map.

9. The system of claim 1, wherein the indication of the configuration of the neurological lead includes at least one of an electrode number, an electrode size, and an electrode position on the neurological lead.

10. The system of claim 1, comprising:
the interface configured to receive an implantation location of the neurological lead, wherein the implantation location of the neurological lead comprises at least one of an implantation depth, implantation coordinates, and an orientation.

11. A method to select stimulation electrodes of an implantable neurostimulation device, comprising:
receiving an indication of a configuration of a neurological lead, the neurological lead comprising a plurality of electrodes;
transmitting, to an implanted stimulation device, a first message to deliver a first stimulation signal with a first set of stimulation parameters to one of the plurality of electrodes;
transmitting, to the implanted stimulation device, a second message to deliver a second stimulation signal with a second set of stimulation parameters to the one of the plurality of electrodes;
receiving an indication of a first stimulation effect on a patient based on the first stimulation signal to the one of the plurality of electrodes;
receiving an indication of a second stimulation effect on the patient based on the second stimulation signal to the one of the plurality of electrodes;
determining a therapeutic window for the one of the plurality of electrodes based on a difference between the first set of stimulation parameters and the second set of stimulation parameters, the indication of the first stimulation effect, and the indication of the second stimulation effect; and
generating a therapeutic window map based on the therapeutic window for the one of the plurality of electrodes and the indication of the configuration of the neurological lead.

12. The method of claim 11, wherein the first stimulation signal has a first current level and the second stimulation signal has a second current level different than the first current level.

13. The method of claim 11, wherein the first stimulation signal has a first voltage level and the second stimulation signal has a second voltage level different than the first voltage level.

14. The method of claim 11, comprising:
transmitting, to the implanted stimulation device, a third message to increase a stimulation parameter to the one of the plurality of electrodes.

15. The method of claim 11, comprising:
determining a benefit score for each of at least the portion of the plurality of electrodes based on the minimum response stimulation parameter; and
determining a side effect score for each of at least the portion of the plurality of electrodes based on the maximum response stimulation parameter.

16. The method of claim 11, comprising:
determining a minimum response stimulation parameter for each of at least a portion of the plurality of electrodes;
determining a maximum response stimulation parameter for each of at least the portion of the plurality of electrodes; and
determining a therapeutic window for each of at least the portion of the plurality of electrodes based on the minimum response stimulation parameter for each of at least the portion of the plurality of electrodes and the maximum response stimulation parameter for each of at least the portion of the plurality of electrodes.

17. The method of claim 16, comprising:
generating the therapeutic window map based on the therapeutic window for each of at least the portion of the plurality of electrodes.

18. The method of claim 16, comprising:
setting at least one of the plurality of electrodes as a stimulation electrode based on the therapeutic window map.

19. The method of claim 11, wherein the indication of the configuration of the neurological lead includes at least one of an electrode number, an electrode size, and an electrode position on the neurological lead.

20. The method of claim 11, comprising:
receiving an implantation location of the neurological lead, wherein the implantation location of the neurological lead comprises at least one of an implantation depth, implantation coordinates, and an orientation.
